(19) United States
(12) Patent Application Publication
(10) Pub. No.: US 2022/0202334 A1
(43) Pub. Date: Jun. 30, 2022

(54) IMAGE-ASSISTED SENSOR SELECTION IN THE CAPACITIVE MEASUREMENT OF BIOELECTRICAL SIGNALS

(71) Applicant: Siemens Healthcare GmbH, Erlangen (DE)

(72) Inventors: Ulrich BATZER, Spardorf (DE); Tobias HEIMANN, Erlangen (DE)

(73) Assignee: Siemens Healthcare GmbH, Erlangen (DE)

(21) Appl. No.: 17/552,432

(22) Filed: Dec. 16, 2021

(30) Foreign Application Priority Data
Dec. 30, 2020 (DE) 10 2020 216 602.1

(51) Int. Cl. A61B 5/277; A61B 5/00; G16H 30/40

(52) U.S. Cl. CPC A61B 5/277; A61B 5/704; A61B 5/7285; A61B 5/0077; A61B 2562/046; A61B 5/721; G16H 30/40; A61B 2562/0214; A61B 5/7267

(57) ABSTRACT

A sensor selection facility is described. In an embodiment, the sensor selection facility includes an image capture unit for acquiring image data from a patient; a position ascertainment unit for ascertaining positions of the capacitive sensor electrodes relative to the body of the patient based upon the image data; an evaluation unit for ascertaining the anticipated quality of a sensor signal from the capacitive sensor electrodes based upon the ascertained positions; and a combination unit for defining a combination strategy for combining the sensor signals from the respective capacitive sensor electrodes based upon the ascertained signal quality of the capacitive sensor electrodes. A differential voltage measurement system is also described. A method and computer readable medium for adapting a differential voltage measurement system are moreover described.

### PRIORITY STATEMENT

The present application hereby claims priority under 35 U.S.C. § 119 to German patent application number DE 102020216602.1 filed Dec. 30, 2020, the entire contents of which are hereby incorporated herein by reference.

### FIELD

Example embodiments of the invention generally relate to a sensor selection facility in a differential voltage measurement system with a signal measurement circuit for measuring bioelectrical signals with a number of wanted signal paths each having a capacitive sensor electrode for acquiring a measurement signal. Example embodiments of the invention also relates to a differential voltage measurement system. Example embodiments of the invention further relates to a medical imaging system. Example embodiments of the invention moreover relates to a method for adapting a differential voltage measurement system.

### BACKGROUND

Voltage measurement systems, in particular differential voltage measurement systems, for measuring bioelectrical signals are used, for example, in medicine for measuring electrocardiograms (ECG), electroencephalograms (EEG) or electromyograms (EMG).

When imaging the heart, there is a need to measure cardiac activity due to the major movement of the heart during a heartbeat. Cardiac activity is measured using sensors which have to be fastened to the patient's body.

The sensors used for the stated measurements are conventionally electrodes which are fastened to the patient's body. An alternative approach which has been investigated for some time is capacitive ECG measurement, in which an ECG signal can also be acquired purely capacitively without the capacitive sensors being in direct contact with the patient. In this manner, it is for example possible to perform an ECG measurement on a clothed patient, wherein the capacitive sensors can be arranged on the clothing.

Electrocardiograms are required for example for measuring cardiac activity in connection with imaging the heart in order to synchronize the major movements of the heart during the heartbeat with image capture.

If a high quality ECG signal is to be obtained with capacitive sensors, the capacitive sensors must be arranged in the immediate vicinity of the patient's body. For example, in the case of a patient lying on their back, the shoulder blades are an appropriate contact region and the buttocks to hip are appropriate measurement points since the stated body parts are firmly pressed by the patient's body weight against the support.

### SUMMARY

However, the inventors have discovered that as a result of the patient's differing physical constitution, in particular in relation to body size, weight and skeletal structure, the stated target regions are differently oriented in each patient. A measurement structure using capacitive sensors therefore requires a plurality of sensors which cover all possible ideal measurement points.

However, the inventors have discovered that once a patient has settled on a patient couch for imaging, it is often difficult quickly and accurately to select those sensors with which high signal quality is achieved.

Test measurements with individual sensors have previously been carried out in order to identify the favorably positioned sensors. However, the inventors have discovered that this procedure is impaired by the fact that, as the patient settles on a patient couch, static discharge effects can occur which take about 30 seconds to completely disappear and, up to this point in time, complicate any analysis of the capacitive sensors' signal quality. Also, the inventors have discovered that analysis of the signals at rest is only of limited use for predicting whether the quality of these signals will also remain high in the event of small patient movements, such as breathing or acceleration of the patient through the imaging system.

As a result of a typical heart rate of about 60 to 80 beats per minute, the inventors have discovered that using the heartbeat as the criterion for good signal quality means that only a few sampled values are available at the start of an examination.

At least one embodiment of the present invention thus enables capacitive differential measurement of bioelectrical signals with enhanced signal quality.

Embodiments are directed to a sensor selection facility, a differential voltage measurement system, a medical imaging system and a method for adapting a differential voltage measurement system.

The sensor selection facility according to at least one embodiment of the invention in a differential voltage measurement system with a signal measurement circuit for measuring bioelectrical signals with a number of wanted signal paths each having a capacitive sensor electrode for acquiring a measurement signal comprises an image capture unit for acquiring patient image data. The image data is preferably acquired from the patient by the image capture unit prior to differential measurement of a bioelectrical signal from the patient. Procedures are, however, also possible, in which images of the patient are repeatedly captured during a differential measurement and the configuration of the capacitive sensor measurements is adapted virtually in real time in order to be able to maintain or keep constant the quality of measurement signals even in dynamic scenarios, for example in which the patient moves more frequently during a measurement or is moved by an apparatus, such as for example a patient couch, and thus also to be able to constantly modify optimum sensor configurations.

The differential voltage measurement system according to at least one embodiment of the invention includes at least one first capacitive sensor or such a sensor electrode and at least one second capacitive sensor or such a second capacitive sensor electrode for measuring bioelectrical measurement signals. Furthermore, the differential voltage measurement system according to the invention preferably includes at least one third capacitive sensor or a third capacitive sensor electrode for equipotential bonding between an object being measured, preferably a patient, and the differential voltage measurement system. A reference common-mode interference signal can also be generated via this third capacitive sensor. The differential voltage measurement system according to the invention also includes a measurement facility. The measurement facility includes a signal measurement circuit for measuring the bioelectrical signals. The differential voltage measurement system according to the invention furthermore includes a signal selection facility according to the invention which can, in a targeted manner, select and/or particularly strongly weight sensor signals from individual capacitive sensor electrodes which enable particularly good signal quality on measurement of bioelectrical signals. The signal selection facility according to the invention can be comprised, for example, by the measurement facility.

The medical imaging system according to at least one embodiment of the invention includes a medical imaging facility, preferably a CT scan unit, the differential voltage measurement system according to at least one embodiment of the invention, preferably together with a synchronization facility for synchronizing an imaging procedure of the medical imaging facility using the measurement signals of the differential voltage measurement system. The medical imaging system according to at least one embodiment of the invention shares the advantages of the sensor selection facility according to at least one embodiment of the invention. The enhanced signal quality of the bioelectrical signals from the patient achieved by the sensor selection facility according to at least one embodiment of the invention of the medical imaging system according to at least one embodiment of the invention brings about, for example on application to the synchronization of imaging with a patient's dynamic physiological behavior, such as for example cardiac motion, more precise or enhanced synchronization of the imaging by a medical imaging facility, for example a CT scan unit or an MR scan unit, with the patient's physiology or indeed for the first time makes such synchronization possible and so makes a major contribution to enhanced image quality of an image capture from a patient by the medical imaging facility.

In the method according to at least one embodiment of the invention for adapting a differential voltage measurement system, image data is acquired by an image capture unit, for example by an imaging camera, from a patient, preferably temporally before the differential measurement of a bioelectrical signal from the patient.

The positions of capacitive sensor electrodes of the differential voltage measurement system relative to the patient's body are furthermore ascertained by the image capture of the patient, which preferably proceeds in advance, i.e. before measurement of the bioelectrical signal. Based upon the ascertained positions, an anticipated quality of the sensor signals from the respective capacitive sensor electrodes is ascertained. Based upon the ascertained anticipated signal quality of the sensor signals from the respective capacitive sensor electrodes, a combination strategy for combining the sensor signals from the respective capacitive sensor electrodes is ascertained and defined. For example, suitable capacitive sensor electrodes at particularly favorable positions are selected based upon the ascertained positions. The method according to at least one embodiment of the invention for adapting a differential voltage measurement system shares the advantages of the sensor selection facility according to at least one embodiment of the invention.

A major part of the above-stated components of the sensor selection facility according to at least one embodiment of the invention, in particular the position ascertainment unit, the evaluation unit and the combination unit, can be embodied entirely or in part in the form of software modules in a processor of a corresponding capacitive differential voltage measurement system. A largely software-based embodiment has the advantage that, optionally after additional retrofitting with hardware elements, such as for example an image capture unit, capacitive differential voltage measurement systems which are already in service can also straightforwardly be retrofitted to operate in the manner according to the invention via a software update.

In this respect, at least one embodiment of the invention is also directed to computer program product with a computer program which is directly loadable into a storage facility of a capacitive differential voltage measurement system, with program parts for carrying out all the steps of the method according to at least one embodiment of the invention when the program is executed in the voltage measurement system. In addition to the computer program, such a computer program product can optionally comprise additional elements such as for example documentation and/or additional components including hardware components, such as for example hardware keys (dongles etc.) for using the software.

A computer-readable medium, for example a memory stick, hard disk or other transportable or permanently installed data storage medium on which are stored the program parts of the computer program which can be read in and executed by a computer unit of the differential voltage measurement system can be used for transport to the differential voltage measurement system and/or for storage on or in the differential voltage measurement system. The computer unit can to this end have, for example, one or more cooperating microprocessors or the like.

At least one embodiment of the invention is also directed to a sensor selection facility in a differential voltage measurement system including a signal measurement circuit for measuring bioelectrical signals with a number of wanted signal paths, including respective capacitive sensor electrodes for acquiring a measurement signal, the sensor selection facility comprising:

an image capture unit to acquire image data from a patient;

a position ascertainment unit to ascertain positions of the capacitive sensor electrodes relative to a body of the patient based upon the image data;

an evaluation unit to ascertain an anticipated quality of the bioelectrical signals from the respective capacitive sensor electrodes based upon the positions ascertained; and

a combination unit to define a combination strategy for combining the bioelectrical signals from the respective capacitive sensor electrodes based upon the anticipated quality of the bioelectrical signals ascertained from the respective capacitive sensor electrodes.

At least one embodiment of the invention is also directed to a differential voltage measurement system, comprising:

at least one first electrode and one second electrode for measuring bioelectrical measurement signals;

a measurement facility including

a signal measurement circuit for measuring the bioelectrical signals, and

the sensor selection facility of an embodiment; and

a drive unit for driving the at least one first electrode and the at least one second electrode for measuring bioelectrical measurement signals according to a combination strategy defined by the sensor selection facility.

At least one embodiment of the invention is also directed to a medical imaging system, comprising:

a medical imaging facility; and

the differential voltage measurement system of of an embodiment.

At least one embodiment of the invention is also directed to a method for adapting a differential voltage measurement system, comprising:

acquiring image data from a patient;

ascertaining respective positions of respective capacitive sensor electrodes of a differential voltage measurement system relative to a body of the patient based upon the image data acquired from the patient;

ascertaining an anticipated signal quality of respective sensor signals from the respective capacitive sensor electrodes based upon the respective positions ascertained; and

defining a combination strategy for combining the respective sensor signals from the respective capacitive sensor electrodes based upon the anticipated signal quality of the respective sensor signals ascertained from the respective capacitive sensor electrodes.

At least one embodiment of the invention is also directed to a non-transitory computer program product with a computer program, directly loadable into a storage facility of a voltage measurement system, including program parts for carrying out the method of an embodiment when the computer program is executed in the voltage measurement system.

At least one embodiment of the invention is also directed to a non-transitory computer-readable medium storing program parts of a computer program, readable in and executable by a computer unit, to carry out the method of an embodiment when the program parts are executed by the computer unit.

### BRIEF DESCRIPTION OF THE DRAWINGS

The invention is explained once more in greater detail below based upon example embodiments with reference to the appended figures. In the various figures, identical components are here provided with identical reference characters.

The figures are not in general to scale. In the figures:

FIG. 1 is a schematic representation of a medical imaging system with a measurement arrangement for the differential measurement of capacitive signals,

FIG. 2 is a schematic representation of a sensor selection facility according to an example embodiment of the invention,

FIG. 3 is a flow chart which schematically represents a method for adapting a differential voltage measurement system according to an example embodiment of the invention,

FIG. 4 is a schematic representation of a computed tomography system according to an example embodiment of the invention,

FIG. 5 is a schematic representation of a patient on a patient couch.

The figures in each case take an ECG measurement system 1 by way of example of a differential voltage measurement system 1 for measuring bioelectrical signals S(k), in this case ECG signals S(k). The invention is, however, not restricted thereto.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The sensor selection facility according to at least one embodiment of the invention in a differential voltage measurement system with a signal measurement circuit for measuring bioelectrical signals with a number of wanted signal paths each having a capacitive sensor electrode for acquiring a measurement signal comprises an image capture unit for acquiring patient image data. The image data is preferably acquired from the patient by the image capture unit prior to differential measurement of a bioelectrical signal from the patient. Procedures are, however, also possible, in which images of the patient are repeatedly captured during a differential measurement and the configuration of the capacitive sensor measurements is adapted virtually in real time in order to be able to maintain or keep constant the quality of measurement signals even in dynamic scenarios, for example in which the patient moves more frequently during a measurement or is moved by an apparatus, such as for example a patient couch, and thus also to be able to constantly modify optimum sensor configurations.

The image capture unit should be taken to mean a unit which produces a projection or spatial representation of the capacitive sensor electrodes arranged on the patient and at least the portion of the patient's body on which are arranged the capacitive sensor electrodes with which a bioelectrical signal, for example an ECG signal, of the patient is to be measured. Optical image capture units, such as for example cameras, video cameras or infrared cameras, are for example suitable for such image representation. Imaging units based on other technologies are, however, also possible. For example, image capture in the course of a prior medical image capture is also possible. In this case, the image capture unit would thus for example be an X-ray imaging unit, preferably a scan unit of a CT system.

The sensor selection facility according to at least one embodiment of the invention also comprises a position ascertainment unit for ascertaining positions of the capacitive sensor electrodes relative to the patient's body based upon the image data. The positions of the capacitive sensor electrodes relative to the body are correlated with a signal quality of the sensor signals from individual capacitive sensor electrodes. Knowledge of the sensor positions can therefore be used to make a prediction as to the anticipated signal quality of a subsequent bioelectrical signal measurement. As will be explained in greater detail below, model-based or AI-based methods can be used for ascertaining this correlation. The position ascertainment unit can be configured to determine the positions of the capacitive sensor electrodes on or relative to the patient in advance, i.e. before measurement of a bioelectrical signal, but it can also be configured to determine the positions of the capacitive sensor electrodes relative to the patient repeatedly, preferably at regular time intervals, during a duration of measurement of bioelectrical signals and a medical imaging procedure preferably clocked therewith in order to take account of patient movements or to maintain measurement signal quality in the event of slippage of the capacitive sensor electrodes due to active or passive patient movement. The capacitive sensor electrodes are preferably fixedly arranged on a patient support, such as for example a foam or textile mat. In this case, the absolute positions of the capacitive sensor electrodes are known. It is then advantageously only necessary to ascertain a position and an orientation of a patient relative to the individual capacitive sensor electrodes by evaluating the image capture in order to be able to ascertain the relative positions of the capacitive sensor electrodes in relation to the patient's body.

The sensor selection facility according to at least one embodiment of the invention additionally comprises an evaluation unit for ascertaining the anticipated quality of one of the sensor signals from the respective capacitive sensor electrodes based upon the ascertained positions, or more specifically the relative positions of the capacitive sensor electrodes in relation to the patient's body.

As will be explained in greater detail below, it is possible to ascertain a relationship between the positions of the capacitive sensor electrodes on the patient's body and a signal quality of the bioelectrical measurement signals measured with the capacitive sensor electrodes, for example based upon a model or of AI.

The sensor selection facility according to at least one embodiment of the invention also comprises a combination unit for defining a combination strategy for combining the sensor signals from the respective capacitive sensor electrodes based upon the ascertained signal quality from the capacitive sensor electrodes.

As will be explained more precisely below, in addition to the quality of the sensor signals from the individual capacitive sensor electrodes, which has for example been ascertained during imaging carried out in advance, the combination strategy can also take account of movement by the patient under examination and/or operating status data of a scan unit of a medical facility, if the measurement of the bioelectrical signals is used by the differential voltage measurement system, for example, to synchronize operation of such a medical facility, for example an imaging facility, with the patient's physiology or the dynamics of the patient's physiology, which is ascertained by the bioelectrical signals.

The sensor signals can be combined both by electrical switching operations and by electronic processing or software-based processing of the acquired sensor signals. In particular, the sensor signals can be combined by selecting particularly favorably positioned capacitive electrodes which are then retained for measurement of a bioelectrical signal. The sensor signals can, however, also be combined by differently weighting the sensor signals from some of the capacitive electrodes or the sensor signals from all available capacitive sensor electrodes. Such weighting can proceed, for example, by software in the course of an evaluation program for the acquired bioelectrical sensor signals.

Therefore, based upon the positions of the capacitive sensors, it is possible, even before a measurement signal or bioelectrical sensor signal is received, to identify which capacitive sensors should best be used for a measurement. The arrangement makes it possible to identify the correct capacitive sensors or sensor electrodes also in the case of unusual heartbeats or heartbeat patterns, which can otherwise be identified only with difficulty, or even during cardiac arrest. The ascertained sensor signal combination can then be transferred to a switching or control facility of the differential voltage measurement system which drives the capacitive sensor electrodes for measuring a bioelectrical signal according to the received combination specification or evaluates the signals thereof accordingly. The image-based, preferably optical, identification of the position of the capacitive sensor electrodes ensures greater tolerance to electrostatic events. Advantageously, a user does not themselves need to know the suitable positions of sensors or is not compelled to select sensors which are suitable for a measurement. The demands placed on a user's expert knowledge are therefore reduced. Advantageously, enhanced signal quality combined with a simplified working procedure for carrying out a differential voltage measurement of a bioelectrical signal from a patient is achieved. In the event of the sensor constellation not offering any appropriate sensors, for example if the sensor electrodes are arranged fixedly on a mat, for a patient's posture and constitution, such a circumstance can be identified and feedback accordingly expedited to the operator. In such a case, the operator knows that they must intervene actively and modify individual parameters which influence measurement quality. For example, the patient has to be requested to reposition themselves or somewhat modify their orientation in space.

Overall, the workflow during a capacitive measurement of bioelectrical signals is thus accelerated and, thanks to the additional feedback, the user feels more secure in handling the new technology of capacitive differential measurement of bioelectrical signals.

As already mentioned above, the differential capacitive voltage measurement system acquires bioelectrical signals for example from a human or animal patient. To this end, it has a number of measurement leads or wanted signal paths. These connect, for example as individual cables, the capacitive sensors which are placed on the patient for acquiring the signals, with the further components of the voltage measurement system, i.e. in particular with the electronics, which serve to evaluate or display the acquired signals.

A person skilled in the art is aware of the fundamental mode of operation of differential voltage measurement systems, for which reason no more detailed explanation is provided at this point. They can in particular take the form of electrocardiograms (ECG), electroencephalograms (EEG) or electromyograms (EMG).

The differential voltage measurement system according to at least one embodiment of the invention includes at least one first capacitive sensor or such a sensor electrode and at least one second capacitive sensor or such a second capacitive sensor electrode for measuring bioelectrical measurement signals. Furthermore, the differential voltage measurement system according to at least one embodiment of the invention preferably includes at least one third capacitive sensor or a third capacitive sensor electrode for equipotential bonding between an object being measured, preferably a patient, and the differential voltage measurement system. A reference common-mode interference signal can also be generated via this third capacitive sensor. The differential voltage measurement system according to at least one embodiment of the invention also includes a measurement facility. The measurement facility includes a signal measurement circuit for measuring the bioelectrical signals. The differential voltage measurement system according to at least one embodiment of the invention furthermore includes a signal selection facility according to at least one embodiment of the invention which can, in a targeted manner, select and/or particularly strongly weight sensor signals from individual capacitive sensor electrodes which enable particularly good signal quality on measurement of bioelectrical signals. The signal selection facility according to at least one embodiment of the invention can be comprised, for example, by the measurement facility.

The voltage measurement system according to at least one embodiment of the invention moreover includes a drive unit for driving the at least one first capacitive sensor electrode and the at least one second capacitive sensor electrode for measuring bioelectrical measurement signals according to the defined combination strategy. Driving can be achieved by, for example, electrically switching, or switching on or off, individual capacitive sensor electrodes, but can also be achieved by subsequent weighted evaluation and combination of the acquired sensor signals from the differently positioned capacitive sensor electrodes or by a combination of the two procedures.

In order to measure a reference common-mode interference signal, the differential voltage measurement system preferably has a third wanted signal path with the already mentioned third capacitive sensor. The differential voltage measurement system preferably further comprises a driver circuit which is connected between a current-measuring resistor and the signal measurement circuit. The driver circuit is also denoted “right leg drive” (RLD) and is responsible for generating a signal which is controlled to the average common-mode voltage of individual or all of the signals. As a result, the measured common-mode interference signals can be reduced in the wanted signal paths.

The third, preferably provided wanted signal path (or “right leg drive path”) ensures equipotential bonding between the patient and the capacitive differential voltage measurement system or the ECG measurement system. The capacitive sensor of the third wanted signal path is preferably placed on the patient's right leg, so explaining the designation “right leg drive”. This third potential can, however, also in principle be acquired at another location on the patient.

The differential voltage measurement system according to at least one embodiment of the invention shares the advantages of the sensor selection facility according to at least one embodiment of the invention. In particular, the quality of the bioelectrical signals generated by the differential voltage measurement system is enhanced by application of the ascertained combination strategy.

The medical imaging system according to at least one embodiment of the invention includes a medical imaging facility, preferably a CT scan unit, the differential voltage measurement system according to at least one embodiment of the invention, preferably together with a synchronization facility for synchronizing an imaging procedure of the medical imaging facility using the measurement signals of the differential voltage measurement system. The medical imaging system according to at least one embodiment of the invention shares the advantages of the sensor selection facility according to at least one embodiment of the invention. The enhanced signal quality of the bioelectrical signals from the patient achieved by the sensor selection facility according to at least one embodiment of the invention of the medical imaging system according to at least one embodiment of the invention brings about, for example on application to the synchronization of imaging with a patient's dynamic physiological behavior, such as for example cardiac motion, more precise or enhanced synchronization of the imaging by a medical imaging facility, for example a CT scan unit or an MR scan unit, with the patient's physiology or indeed for the first time makes such synchronization possible and so makes a major contribution to enhanced image quality of an image capture from a patient by the medical imaging facility.

In the method according to at least one embodiment of the invention for adapting a differential voltage measurement system, image data is acquired by an image capture unit, for example by an imaging camera, from a patient, preferably temporally before the differential measurement of a bioelectrical signal from the patient.

The positions of capacitive sensor electrodes of the differential voltage measurement system relative to the patient's body are furthermore ascertained by the image capture of the patient, which preferably proceeds in advance, i.e. before measurement of the bioelectrical signal. Based upon the ascertained positions, an anticipated quality of the sensor signals from the respective capacitive sensor electrodes is ascertained. Based upon the ascertained anticipated signal quality of the sensor signals from the respective capacitive sensor electrodes, a combination strategy for combining the sensor signals from the respective capacitive sensor electrodes is ascertained and defined. For example, suitable capacitive sensor electrodes at particularly favorable positions are selected based upon the ascertained positions. The method according to at least one embodiment of the invention for adapting a differential voltage measurement system shares the advantages of the sensor selection facility according to at least one embodiment of the invention.

A major part of the above-stated components of the sensor selection facility according to at least one embodiment of the invention, in particular the position ascertainment unit, the evaluation unit and the combination unit, can be embodied entirely or in part in the form of software modules in a processor of a corresponding capacitive differential voltage measurement system. A largely software-based embodiment has the advantage that, optionally after additional retrofitting with hardware elements, such as for example an image capture unit, capacitive differential voltage measurement systems which are already in service can also straightforwardly be retrofitted to operate in the manner according to the invention via a software update.

In this respect, at least one embodiment of the invention is also directed to computer program product with a computer program which is directly loadable into a storage facility of a capacitive differential voltage measurement system, with program parts for carrying out all the steps of the method according to at least one embodiment of the invention when the program is executed in the voltage measurement system. In addition to the computer program, such a computer program product can optionally comprise additional elements such as for example documentation and/or additional components including hardware components, such as for example hardware keys (dongles etc.) for using the software.

A computer-readable medium, for example a memory stick, hard disk or other transportable or permanently installed data storage medium on which are stored the program parts of the computer program which can be read in and executed by a computer unit of the differential voltage measurement system can be used for transport to the differential voltage measurement system and/or for storage on or in the differential voltage measurement system. The computer unit can to this end have, for example, one or more cooperating microprocessors or the like.

Further, particularly advantageous refinements and further developments of the invention are revealed by the claims and by the following description, wherein the claims of one category of claim can also be further developed in a manner similar to the claims and passages of the description relating to another category of claim and in particular individual features of different example embodiments or variants can also be combined to form new example embodiments or variants.

The combination unit is preferably configured to select and switch on capacitive sensor electrodes based upon the ascertained anticipated quality of a sensor signal of the individual capacitive sensor electrodes. In other words, the capacitive sensor electrodes which, based upon their position relative to the patient's body, are to be expected to have particularly good signal quality are selected as the measuring electrodes or at least their signal is particularly strongly weighted on signal evaluation. Advantageously, signal interference can be eliminated or reduced from signals in advance, i.e. before the actual differential measurement, such that the actual differential measurement can proceed with enhanced, preferably high quality.

The image capture unit of the sensor selection facility according to at least one embodiment of the invention preferably has a 3D camera. A 3D camera makes it possible to process depth information from image data in order in this manner to obtain more precise position information for individual sensors and thus, in comparison with the use of a 2D camera, enhanced data acting as the basis for an assessment of a sensor contact's quality.

The position ascertainment unit of the sensor selection facility according to at least one embodiment of the invention is particularly preferably configured to ascertain anatomical landmarks on the patient's body based upon the image data and to ascertain the positions of the capacitive sensor electrodes based upon the known positions of the landmarks. Particularly conspicuous points on the body or body parts which can be recognized on the images of the image capture unit are advantageously used as reference points for determining the position of the sensor electrodes.

The combination unit of the sensor selection facility according to at least one embodiment of the invention is preferably configured to combine the signals from the capacitive sensor electrodes based upon the ascertained signal quality of the capacitive sensor electrodes in such a manner that a combined aggregate signal has a predetermined minimum signal quality. Based upon the position of the individual sensor electrodes, a forecast with regard to the quality of a combined signal from a plurality of sensor electrodes is advantageously made, for example based upon a model or by application of AI systems. Based upon a minimum quality which is defined in advance, a set of “permitted combinations” of sensor electrodes can be ascertained and possible sensor combinations can be selected from this set, for example taking additional criteria into account.

The combination unit of the sensor selection facility according to at least one embodiment of the invention is particularly preferably configured to combine the signals from the capacitive sensor electrodes in such a manner that an aggregate signal exhibits an optimum signal quality. In this variant, it is advantageously attempted to achieve the best possible signal quality which can be expected.

The combination unit of the sensor selection facility according to at least one embodiment of the invention is likewise preferably configured to produce the aggregate signal by combining the sensor signals from the respective capacitive sensor electrodes in a weighted manner. In this variant, the signal can advantageously take account of a plurality, possibly even all, of the sensor electrodes, wherein an enhanced signal quality of the aggregate signal is achieved by increased weighting of higher quality signal components.

The combination unit of the sensor selection facility according to at least one embodiment of the invention can also particularly preferably comprise an artificial intelligence-based analysis unit which is configured to ascertain a combination of the sensor signals from the respective capacitive sensor electrodes based upon an algorithm based on machine learning. An algorithm based on machine learning is here preferably trained such that, based upon the current image of the image capture unit, it combines the signals from the individual capacitive sensor electrodes in such a manner that an enhanced, preferably optimum, signal quality of an aggregate signal is achieved. Patient movements can be identified by the image capture unit and the optimum weighting or selection of the sensor electrodes during an imaging operation updated accordingly, whereby artifacts which occur in the measurement signal curves are suppressed.

In comparison with a rigid model, an AI-based combination of sensor signals can advantageously be put to particularly flexible use. The flexibility of capacitive sensors is therefore linked with the robust signal reconstruction of machine learning in order to achieve higher measurement signal quality combined with a simplified working procedure.

For example, statistics based on numerous human test subjects and test runs can be used as the basis for assigning an expected value for a signal quality value to each possible position of a sensor relative to the patient's body and for creating a database. Intermediate values or intermediate positions or corresponding intermediate orientations of the sensors relative to the patient's body which are not included in the database can optionally be interpolated. The database can also be used for training an artificial neural network or an artificial intelligence system or as the basis for modeling a relationship between the orientation and position of a patient's body relative to the capacitive sensor electrodes and a signal quality.

The combination unit of the sensor selection facility according to at least one embodiment of the invention can alternatively or additionally also comprise a modeling unit which is configured to ascertain a combination of the sensor signals from the respective capacitive sensor electrodes based upon a model which is to be parameterized. In comparison with an AI-based scenario, such a procedure may be particularly economical in time and resources, if a relationship between signal quality and relative position of the sensors in relation to a patient's body can be particularly well and simply modeled by a theoretically and/or experimentally ascertained model.

In order to ascertain the relative positions of the capacitive sensor electrodes in relation to the patient's body, the patient is located on a sensor mat having a plurality of capacitive sensor electrodes, which are distributed for example in a grid on the sensor mat. The position of the sensor electrodes on the sensor mat is preferably fixed and thus also known. If, using an image capture unit, for example a stereo camera, an image of the patient is now captured before a differential measurement of bioelectrical signals, the patient's contour, for example the patient's outline, and landmarks and the outline of the sensor mat and optionally the position of at least some of the sensors which are not covered up by the patient can be extracted from the image. Landmarks may also be supplemented or made more readily visible and extractable by additional markers positioned on the patient.

Based upon the patient's outline in the image or based upon landmarks on the image, it is then possible to ascertain the relative position of the individual sensor electrodes in relation to the patient's individual body parts, such as for example the patient's upper back, pelvis or buttocks. Once the relative positions of the individual sensor electrodes in relation to individual body parts are known, it is then possible, for example based upon a model or a machine learning-based algorithm, to ascertain an anticipated signal quality of the individual sensor electrodes. The model or AI-based algorithm may also include further data, such as for example information regarding body weight, BMI, or the distribution of fat or muscle in the body, in order to forecast the signal quality of the individual sensor electrodes. To this end, a contact pressure distribution of the patient's body on the sensor mat can be ascertained and then, as a function of location-dependent contact pressure and of the relative positions of the sensor electrodes, the signal quality of the measurement signals of the individual sensor electrodes can be ascertained or forecast. As a function thereof, suitable sensor electrodes can be selected for a measurement or their signals can be correspondingly weighted in order to optimize the signal quality of the aggregate signal or at least enhance it to a predetermined minimum quality.

Training data for an AI-based algorithm can, for example, comprise image data or outline data, in each case in vector form, as input data, and the signal quality values assigned to individual sensor electrodes or direct instructions to select individual sensor electrodes as output data. A neural network is trained with the training data until it outputs the already known initial values of the training data. The training data can be obtained, for example, from a database containing the corresponding data for a plurality of examinations from the past. In addition to the relative positions of the sensors in relation to the patient's body, input data can also include values for the operating status of a scan unit, such as for example the rotation of the detector or the timing of triggering X-rays in X-ray imaging, in order to be able simultaneously to suppress any resultant artifacts. A relative position of the patient couch or of the patient in relation to an X-ray source can also be included as an input value. This is because interference effects caused by X-rays conventionally occur only in the X-ray source's current irradiation zone. If this zone is known, it is possible to switch over to sensor electrodes which are not affected by or not exposed to the X-rays in order to enhance signal quality. Other known or measurable interference effects, such as for example electric fields or muscle tremor, can also be included in modeling or training of an AI-based algorithm for selecting sensor electrodes or for determining signal quality parameters, in order also to take account of or suppress these effects in signal generation and so further enhance the signal quality of a bioelectrical measurement signal.

Time series data of input parameters can also be used to train the analysis unit or to adapt the model of the modeling unit of the sensor selection facility according to at least one embodiment of the invention. Time series data can make sensor selection more robust, for instance outliers/interference can be identified by comparing current sensor values or signal values with previous values. The sensors affected by the interference can then be temporarily excluded from the selection or be interpolated by adjacent sensors, so that the reconstruction of the aggregate signal is not impaired.

The combination unit of the sensor selection facility according to at least one embodiment of the invention can particularly preferably be configured to determine the combination strategy based upon one of the following items of information:

movements of the patient,

operating status data of a scan unit of a medical facility.

Advantageously, it is for example possible dynamically to adapt a combination of sensor electrodes during a measurement or an imaging procedure which is preferably synchronized with a patient's physiology to a patient's movements, such that the measurement does not have to be repeated because the patient has inadvertently moved.

Advantageously, an interference effect as a consequence of X-rays can for example be taken into account by taking account of the operating status of the medical imaging facility, in particular of a CT device. For example, in a CT capture, an X-ray source rotates around the patient, such that the patient is periodically irradiated from different directions. As a result, the interference profile of the individual sensor electrodes likewise varies periodically. Advantageously, by taking account of such medical imaging facility parameters, it is now possible periodically and dynamically to adapt the selection of suitable sensor electrodes to the current status of the medical imaging facility, since the movement of the X-ray source is known and predictable.

Another movement of a medical imaging facility to be taken into account in the selection of the capacitive sensor electrodes can be a movement of a patient table which results in movement of the patient's body and thus of the sensor electrodes relative to an interference source, such as for example a zone exposed to X-rays. Advantageously, such a change can also be taken dynamically into account in the selection of suitable sensor electrodes, since the movement of the patient table is known in advance and is predictable.

As has already been mentioned, an interference signal acquisition facility, which comprises a driver circuit for the right-hand leg, can also be part of the differential voltage measurement system according to at least one embodiment of the invention. Using such a driver circuit for the right leg, interference arising from an electrical field and acting directly on the patient can be ascertained by measuring an “RLD current” and included in the interference suppression.

An interference signal acquisition facility of the differential voltage measurement system according to at least one embodiment of the invention can also comprise a shielding current measurement unit for measuring shielding currents which is configured to acquire the entire shielding current or the shielding current per capacitive sensor. Such shielding currents occur on the capacitive sensor shielding. This shielding current is preferably measured at a connection of the shielding of the individual capacitive sensors to a GND (ground) potential. The shielding currents can be measured individually for each sensor cable connection, but they can also be measured at a common measurement point if each shield is set to ground potential via the measurement point. The shielding currents can also be measured at the shields of the sensor cable connection or between the capacitive sensors and their input buffers or between the input buffers and the subsequent units of the capacitive differential measurement system.

Advantageously, this variant also takes account of interference effects on the measurement signal which are caused by currents on the shielding of the capacitive sensors and their cable connection and possibly occur at all the sensor cable connections, such that it would not be possible to compensate for them solely by selecting individual sensors. If the stated interference signals are ascertained by the interference signal acquisition facility, they can be suppressed by a suitable compensating facility in order to enhance measurement signal quality in this way.

The interference signal acquisition facility of the differential voltage measurement system according to at least one embodiment of the invention can also comprise a ground current measurement unit for measuring ground currents. A ground current flows from the medically insulated protected patient side to the units of the capacitive differential measurement system which handle evaluation. A measurement point should preferably be positioned between the GND of the measurement circuit and the GND of the further processing circuit. The measurement circuit comprises the medically protected patient part, while the further processing circuit comprises the unit which handles evaluation. In functional terms, the measurement circuit is conventionally the circuit arrangement including HW filters and ESD protection up to and including the AD converter, while the further processing circuit starts from the AD converter. Advantageously, the signal quality of a bioelectrical signal can likewise be enhanced by compensating interference by such a ground current.

Advantageously, it is thus also possible according to at least one embodiment of the invention to combine the selection of sensor electrodes and/or the weighting of sensor signals with compensation of interference signals based upon a measurement of these interference signals at the individual capacitive sensor electrodes in order further to enhance the quality of the measurement signals from a differential measurement of bioelectrical signals. In this context, reference is made to patent applications with the official file references 10 2020 214 191.6, 10 2020 214 183.5, 20 2020 101 579.6 and 19218075.0 which relate in detail primarily to the measurement and compensation of interference signals in the differential measurement of bioelectrical signals and which are to be included in the present patent application by this reference.

It should additionally be noted at this point that the differential voltage measurement system according to at least one embodiment of the invention or the sensor selection facility according to at least one embodiment of the invention can also be used for monitoring the health status of people in means of transport, in particular in motor vehicles. Advantageously, the enhanced signal quality in the measurement of physiological measurement data, for example a heart rate or an ECG curve of an occupant, in particular of a driver, enhances the reliability of monitoring the health and fitness to drive of the occupant, such that health protection and traffic safety is increased for everyone concerned.

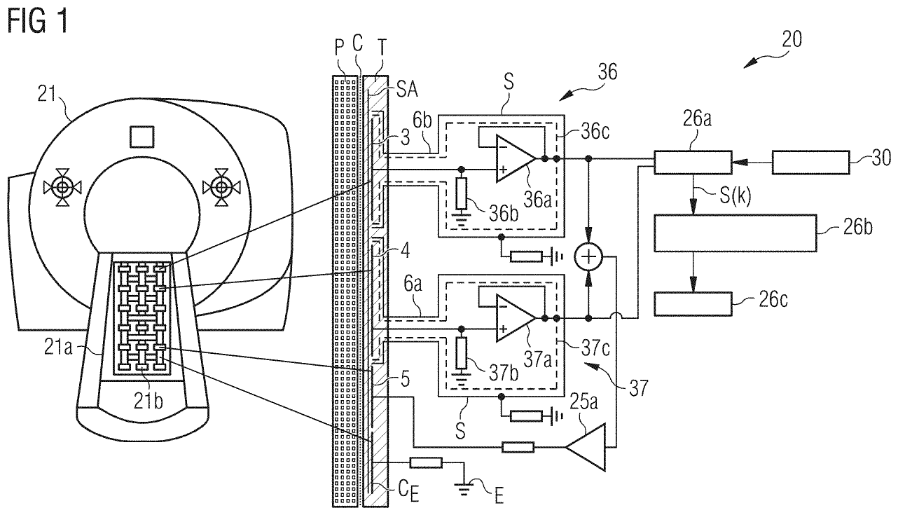

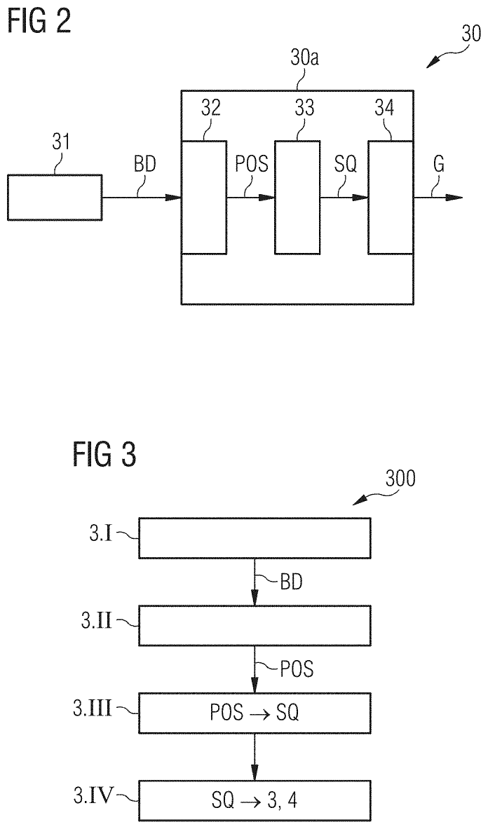

FIG. 1 is a schematic representation of an arrangement with a capacitive differential measurement system 20 according to an example embodiment of the invention and a scan unit 21 of a CT system. The scan unit 21 comprises a patient couch 21a on which a patient P is placed for an examination. On the patient couch 21a there is for example a mat 21b with a plurality of capacitive sensors 3, 4, 5 for ECG measurement during an imaging procedure. Such a mat 21b usually comprises a foam, such as for example polyurethane, and a coating with films. The capacitive sensors 3, 4, 5 are shown once again on the right-hand side of the figure. The capacitive sensors 3, 4, 5 are incorporated into a mat material T which is transparent to X-rays. The mat 21b is in contact with the clothing C of the patient P. The patient P or the patient's body P is merely symbolized as a rectangular area. The capacitive sensors 3, 4, 5 are additionally protected by a sensor cover SA which separates the capacitive sensors 3, 4, 5 from the clothing C of the patient P. FIG. 2 shows a total of three capacitive sensors or sensor electrodes 3, 4, 5 and a capacitive ground connection CE in the lowermost position; the two top and second to top sensors 3, 4 are used as capacitive sensors for measuring a differential capacitive ECG signal and a third capacitive sensor 5 is positioned on the right leg of the patient P in order to form an RLD path. A fourth capacitor CE, which is shown right at the bottom in FIG. 2, is used as a capacitive ground connection between the patient P and ground E.

The two differential capacitive sensors 3, 4 are in each case electrically connected to an amplifier circuit 36, 37 which is denoted input buffer. The amplifier circuit 36 assigned to the first capacitive sensor 3 comprises an operational amplifier 36a and a resistor 36b connected to ground upstream from the positive input of the operational amplifier 36a. The sensor line 6b extending from the sensor 3 to the operational amplifier 36a and the measurement circuit 36 comprising the operational amplifier 36a are surrounded by an “active guard” 36c and shielding S.

The operational amplifier 36 is configured as a “tracking” amplifier. In other words, the negative input of the operational amplifier 36 (characterized with a minus sign) is coupled to the output of the operational amplifier 36. In this manner, a high virtual input impedance at the positive input is achieved for the operational amplifier 36. This means that, due to the voltage matching between the output and the positive input of the operational amplifier 36 (characterized with a plus sign), virtually no current flows between the sensor 3 and the active guard 36c. The positive input of the operational amplifier 36a is furthermore maintained at an electrical bias voltage by a resistor 36b connected to the measuring device ground (also denoted “measurement ground” or GND). The positive input of the operational amplifier 36a can thus be set to a desired measurement potential. DC components are suppressed in this manner. This is desired because the first capacitive sensor 3 is primarily intended to couple capacitively and a varying potential should be avoided. The amplifier circuit 37 of the second capacitive sensor 4 is also constructed in a manner similar to the amplifier circuit 36 of the first capacitive sensor 3. The measurement signal S(t) stored in the amplifier circuits 36, 37 used as an input buffer is digitized by an AD converter (not shown) and transmitted via a switching matrix 26a to an analysis and compensation unit 26.

A third capacitive sensor 5 is configured as a reference sensor or “right leg sensor” for the patient. Equipotential bonding between the patient P and the measurement circuit 20 is created with the assistance of the third capacitive sensor 5. To this end, the output signals from the first and the second input buffers 36, 37 are summed and supplied to an amplifier 25a. This amplifier 25a functions as a neutral driver for an RLD measurement arrangement. A common-mode voltage, which is applied to the inputs of the amplifier 25a, is acquired with this amplifier 25a. The acquired voltage is applied to the input of the amplifier 25a which is configured as an integrating amplifier. The integrating amplifier 25a completes a feedback loop which supplies the patient P with a current IRLD in order to reduce the change in common-mode input voltage for the amplifiers 36, 37 and so for example damp a 60 Hz common-mode voltage.

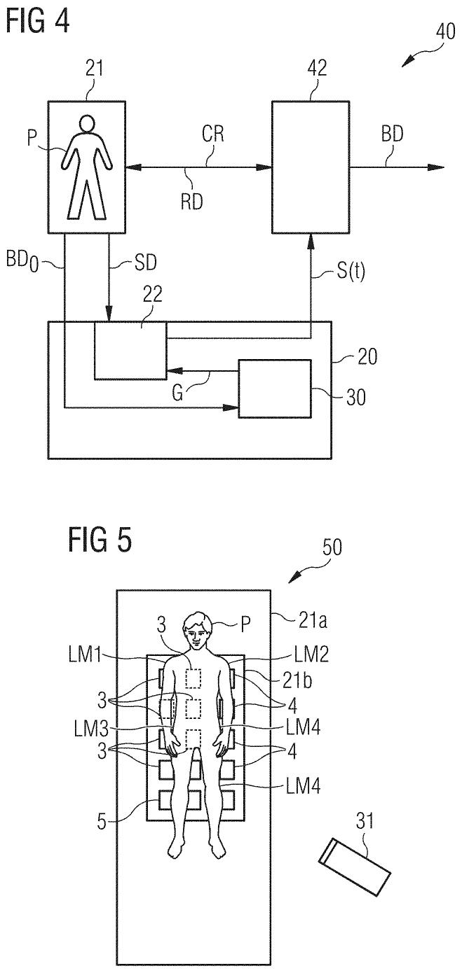

The measurement signal S(k) can be used, for example, for a trigger signal of the CT system 21 in order to synchronize imaging with the patient's cardiac motion. FIG. 4 shows an arrangement which is suitable for this purpose. A sensor selection facility 30 according to at least one embodiment of the invention, as shown in FIG. 2, can be electrically connected for example to the switching matrix 26a. The sensor selection facility 30 ascertains sensor electrodes 3, 4 which generate measurement signals of particularly high quality and drives the switching matrix 26a in such a manner that the sensor electrodes 3, 4 selected for measurement of bioelectrical signals are electrically connected to the analysis unit 26 or are interconnected therewith.

FIG. 2 is a schematic representation of a sensor selection facility 30 according to an example embodiment of the invention. The sensor selection facility 30 comprises an image capture unit 31, in this specific example embodiment a 3D camera, for acquiring image data BD from a patient. The position and orientation of the patient's body and optionally the position of individual capacitive sensor electrodes 3, 4 of an ECG measurement system relative to the body are recorded on the image data BD. Based upon the image data BD, the individual capacitive sensor electrodes 3, 4 or their positions relative to the patient's body are identified with the assistance of image recognition software by a position ascertainment unit 32, which is likewise part of the sensor selection facility 30 and is arranged in an analysis unit 30a of the sensor selection facility 30. The position ascertainment unit 32 moreover ascertains the positions POS of the capacitive sensor electrodes 3, 4 relative to the patient's body. Location finding proceeds based upon landmarks on the patient's body, which are for example represented by body parts, such as the shoulders and pelvis. The positions of the landmarks are for example ascertained relative to the known positions of the sensors, which are for example fixedly arranged on a mat. In this manner, a relative position is ascertained of individual regions of the patient's body in relation to the individual sensor electrodes 3, 4 or vice versa of the sensor electrodes 3, 4 in relation to the regions of the body. The ascertained positions POS of the capacitive sensor electrodes 3, 4 are transmitted to an evaluation unit 33 which, based upon the positions POS of the capacitive sensor electrodes 3, 4 relative to the individual regions of the patient's body, ascertain an anticipated quality SQ of a sensor signal S(k) from the respective capacitive sensor electrodes 3, 4. For example, there are regions of the patient's body against which a capacitive sensor electrode 3, 4 can rest better than in other regions. Therefore, depending on the position of a capacitive electrode 3, 4 relative to the patient's body, it is possible to conclude as to the subsequent signal quality of a measurement signal s(k) from the respective capacitive sensor electrode 3, 4. The anticipated signal quality SQ assigned in each case to a capacitive sensor electrode 3, 4 is transmitted to a combination unit 34. Based upon the ascertained signal quality SQ of the individual capacitive sensor electrodes 3, 4, the combination unit 34 defines a weighting G of the individual measurement signals from the capacitive sensor electrodes 3, 4 differently positioned on or relative to the patient's body. The ascertained weighting G is transferred for example to a drive unit 22 (see FIG. 4) of a differential voltage measurement system 20 (see FIG. 4).

FIG. 3 shows a flow chart 300 which illustrates a method for selecting capacitive measuring electrodes for measuring a biological measurement signal S(t) of a differential voltage measurement system. Image data BD is acquired by a camera from a patient P in step 3.I. The image data BD is used in step 3.II to ascertain positions POS of the capacitive sensor electrodes on or relative to the patient's body. An anticipated quality SQ of the sensor signals from the respective capacitive sensor electrodes 3, 4 is ascertained in step 3.III based upon the ascertained positions POS. Finally, in step 3.IV, suitable capacitive sensor electrodes 3, 4 are selected based upon the ascertained signal quality SQ of the sensor signals from the respective capacitive sensor electrodes 3, 4.

FIG. 4 is a schematic representation of a computed tomography system 40, or CT system for short, according to an example embodiment of the invention. The CT system 40 comprises a scan unit 21 which is used for capturing an image from a patient P. The CT system 40 furthermore comprises a control unit 42 which is used for driving the scan unit 21 with the assistance of control commands CR and for processing the raw data RD acquired from the scan unit 21 and for reconstructing image data BD based upon the acquired raw data RD. The CT system 40 additionally comprises an ECG measurement system 20, which is constructed in the manner according to at least one embodiment of the invention, i.e. in particular comprises the sensor selection facility 30 shown in FIG. 3. Using the image capture unit 31 shown in FIG. 2 (not shown in FIG. 4), for example an imaging camera, the sensor selection facility 30 acquires in advance image data BD0 from the patient P and the arrangement of capacitive sensor electrodes on the patient's body P. As has already been mentioned, the sensor selection facility 30 ascertains in one development of at least one embodiment of the invention a weighting G of the individual capacitive sensor electrodes for a subsequent ECG measurement. The ECG measurement system 20 in particular comprises a drive unit 22 which, during an ECG measurement, receives the sensor signals SD from the individual capacitive sensor electrodes of the ECG measurement system 20 and, in accordance with the weighting G ascertained by the sensor selection facility 30, weights and combines, for example simply sums, these sensor signals SD. A sensor signal S(t) combined in this manner comprises an ECG signal which is optimized or at least enhanced in quality. Weighting G of the sensor signals from the capacitive sensor electrodes can in a borderline case for weights exclusively with the values 0 and 1 become a selection of individual capacitive sensor electrodes 3, 4 which are arranged particularly favorably relative to the body. In such a case, the drive unit 22 switches on only the selected sensor electrodes for acquiring the sensor signals while the remaining capacitive sensor electrodes are in contrast switched off during the ECG measurement. Combinations of these two variants are also possible in which individual capacitive electrodes are switched off and the signals from the switched on capacitive sensor electrodes are differently weighted.

The ECG measurement system 20 measures a heartbeat SD of the patient P and the enhanced quality ECG signal S(t) generated during measurement of the heartbeat is transmitted to the control unit 42 which, by appropriate control commands CR, synchronizes image capture by the scan unit 21 with the heartbeat of the patient P.

FIG. 5 is a schematic representation 50 of a patient P on a patient couch 21a. On the patient couch there is a sensor mat 21b with a plurality of capacitive sensor electrodes 3, 4, 5, which are arranged in a grid on the sensor mat 21b. The patient P is lying with their torso and in part with their legs on the sensor mat 21b. The sensor electrodes 3, 4, 5 are fixedly arranged on the sensor mat 21b and their position is therefore also known in advance. If, using a stereo camera 31a, which is shown at the bottom right of FIG. 5, an image BD0 of the patient P is captured before an ECG measurement, it is for example possible to extract the outline of the patient P and landmarks LM1, LM2, LM3, LM4 and the outline of the sensor mat 21b from the image.

Based upon the outline of the patient P in the image BD0 or based upon landmarks LM1, LM2, LM3, LM4 on the image, it is then possible to ascertain the relative position of the individual sensor electrodes 3, 4, 5 in relation to the individual body parts of the patient P, such as for example the upper back, pelvis or buttocks of the patient P. Once the relative positions of the individual sensor electrodes 3, 4, 5, in relation to individual body parts are known, it is then possible, for example based upon a model or a machine learning-based algorithm, to ascertain an anticipated signal quality SQ of the individual sensor electrodes 3, 4, 5. Further data, such as for example information regarding body weight, BMI, or the distribution of fat or muscle in the body, can also be included in the model, for example to ascertain a contact pressure distribution of the body of the patient P on the sensor mat 21b and, as a function of the location-dependent contact pressure and of the relative positions of the sensor electrodes 3, 4, 5, to forecast a signal quality SQ and, as a function thereof, to select suitable sensor electrodes for measuring ECG signals or suitably to weight the measurement signals acquired from the sensor electrodes in order to achieve signal quality which is enhanced or required for medical purposes.

Training data for an AI-based algorithm can, for example, comprise image data or outline data, in each case in vector form, as input data, and the signal quality values assigned to individual sensor electrodes as output data. A neural network is then trained with the training data until it outputs the already known initial values. In addition to the relative positions, input data can also include values for the operating status of a scan unit 21, such as for example the rotation of the detector and the timing of triggering X-rays, in order to be able simultaneously to suppress artifacts. A relative position of the patient couch 21b or of the patient P in relation to an X-ray source can also be included as an input value. This is because interference effects caused by X-rays conventionally occur only in the X-ray source's current irradiation zone. If this zone is known, sensor electrodes 3, 4, 5 which are not affected by the X-rays may be used for a differential measurement in order to enhance signal quality SQ.

It should finally once again be noted that the apparatuses and methods described in detail above are merely example embodiments which can be modified in the most varied manner by a person skilled in the art without departing from the scope of the invention. The differential voltage measurement system may accordingly not only be an ECG device, but also other medical devices with which bioelectrical signals are acquired, such as for example EEG, EMG etc. While the invention is indeed primarily described with a focus on the synchronization of CT imaging with a patient's heartbeat, there are also many other conceivable applications of the invention, such as for example monitoring the health status of people in means of transport, in particular in motor vehicles. Furthermore, use of the indefinite article “a” does not rule out the possibility of a plurality of the features in question also being present. Likewise, the term “unit” does not rule out the possibility of the latter consisting of a plurality of components which may optionally also be spatially distributed.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A sensor selection facility in a differential voltage measurement system including a signal measurement circuit for measuring bioelectrical signals with a number of wanted signal paths, including respective capacitive sensor electrodes for acquiring a measurement signal, the sensor selection facility comprising:
an image capture unit to acquire image data from a patient;
a position ascertainment unit to ascertain positions of the capacitive sensor electrodes relative to a body of the patient based upon the image data;
an evaluation unit to ascertain an anticipated quality of the bioelectrical signals from the respective capacitive sensor electrodes based upon the positions ascertained; and
a combination unit to define a combination strategy for combining the bioelectrical signals from the respective capacitive sensor electrodes based upon the anticipated quality of the bioelectrical signals ascertained from the respective capacitive sensor electrodes.

2. The sensor selection facility of claim 1, wherein the combination unit is further configured to select and switch on the respective capacitive sensor electrodes based upon the respective anticipated quality of the respective bioelectrical signal ascertained from the respective individual capacitive sensor electrodes for a measurement of bioelectrical signals.

3. The sensor selection facility of claim 1, wherein the image capture unit includes a 3D camera.

4. The sensor selection facility of claim 1, wherein the position ascertainment unit is further configured to ascertain anatomical landmarks on the body of the patient based upon the image data and to ascertain the position of the capacitive sensor electrodes relative to the body of the patient based upon the known position of the landmarks.

5. The sensor selection facility of claim 1, wherein the combination unit is further configured to combine the biometrical signals from the capacitive sensor electrodes based upon the signal quality of the capacitive sensor electrodes ascertained in such a manner that a combined aggregate signal has a minimum signal quality.

6. The sensor selection facility of claim 1, wherein the combination unit is further configured to combine the biometrical signals from the capacitive sensor electrodes in such a manner that an aggregate signal comprises an optimum signal quality.

7. The sensor selection facility of claim 1, wherein the combination unit is further configured to generate the aggregate signal by combining the biometrical signals from the respective capacitive sensor electrodes in a weighted manner.

8. The sensor selection facility of claim 1, wherein the combination unit comprises at least one of:
an artificial intelligence-based analysis unit configured to ascertain a combination of the biometrical signals from the respective capacitive sensor electrodes based upon an algorithm based on artificial intelligence, and
a modeling unit configured to ascertain a combination of the biometrical signals from the respective capacitive sensor electrodes based upon a model which is to be parameterized.

9. The sensor selection facility of claim 8, wherein time series data of input parameters are used to train the artificial intelligence-based analysis unit or to adapt the model of the modeling unit.

10. The sensor selection facility of claim 1, wherein the combination unit is further configured to ascertain the combination strategy based upon one of:
movements of the patient, and
operating status data of a scan unit of a medical imaging facility.

11. A differential voltage measurement system, comprising:
at least one first electrode and one second electrode for measuring bioelectrical measurement signals;
a measurement facility including
a signal measurement circuit for measuring the bioelectrical signals, and
the sensor selection facility of claim 1; and
a drive unit for driving the at least one first electrode and the at least one second electrode for measuring bioelectrical measurement signals according to a combination strategy defined by the sensor selection facility.

12. A medical imaging system, comprising:
a medical imaging facility; and
the differential voltage measurement system of claim 11.

13. A method for adapting a differential voltage measurement system, comprising:
acquiring image data from a patient;
ascertaining respective positions of respective capacitive sensor electrodes of a differential voltage measurement system relative to a body of the patient based upon the image data acquired from the patient;
ascertaining an anticipated signal quality of respective sensor signals from the respective capacitive sensor electrodes based upon the respective positions ascertained;
defining a combination strategy for combining the respective sensor signals from the respective capacitive sensor electrodes based upon the anticipated signal quality of the respective sensor signals ascertained from the respective capacitive sensor electrodes.

14. A non-transitory computer program product with a computer program, directly loadable into a storage facility of a voltage measurement system, including program parts for carrying out the method of claim 13 when the computer program is executed in the voltage measurement system.

15. A non-transitory computer-readable medium storing program parts of a computer program, readable in and executable by a computer unit, to carry out the method of claim 13 when the program parts are executed by the computer unit.

16. The sensor selection facility of claim 2, wherein the image capture unit includes a 3D camera.

17. The sensor selection facility of claim 2, wherein the position ascertainment unit is further configured to ascertain anatomical landmarks on the body of the patient based upon the image data and to ascertain the position of the capacitive sensor electrodes relative to the body of the patient based upon the known position of the landmarks.

18. The sensor selection facility of claim 2, wherein the combination unit is further configured to combine the biometrical signals from the capacitive sensor electrodes based upon the signal quality of the capacitive sensor electrodes ascertained in such a manner that a combined aggregate signal has a minimum signal quality.

19. The sensor selection facility of claim 2, wherein the combination unit is further configured to combine the biometrical signals from the capacitive sensor electrodes in such a manner that an aggregate signal comprises an optimum signal quality.

20. The sensor selection facility of claim 2, wherein the combination unit is further configured to generate the aggregate signal by combining the biometrical signals from the respective capacitive sensor electrodes in a weighted manner.

21. The medical imaging system of claim 12, further comprising:
a synchronization facility for synchronizing an imaging procedure of the medical imaging facility using the measurement signals from the differential voltage measurement system.
